(19) United States
(12) Patent Application Publication
(10) Pub. No.: US 2022/0133176 A1
(43) Pub. Date: May 5, 2022

(54) SYSTEM, METHOD AND APPARATUS FOR DIAGNOSIS AND THERAPY OF NEUROMUSCULAR OR NEUROLOGICAL DEFICITS

(71) Applicant: MindMaze Holding SA, Lausanne (CH)

(72) Inventors: Tej TADI, Lausanne (CH); Aurelien DA CAMPO, Lausanne (CH); Frederic CONDOLO, Lausanne (CH)

(21) Appl. No.: 17/369,446

(22) Filed: Jul. 7, 2021

Related U.S. Application Data
(63) Continuation of application No. 15/849,744, filed on Dec. 21, 2017, now abandoned.
(60) Provisional application No. 62/574,788, filed on Oct. 20, 2017.
(60) Provisional application No. 62/440,481, filed on Dec. 30, 2016.

(51) Int. Cl. A61B 5/11; G16H 20/70; G16H 50/20; G16H 40/63; G16H 40/40; G16H 20/30; A61B 5/00

(52) U.S. Cl. CPC A61B 5/1125; A61B 5/1123; G16H 50/20; G16H 40/63; G16H 40/40; G16H 20/30; A61B 5/1128; A61B 5/4082; A61B 5/0077; A61B 2562/226; A61B 2560/0475; A61B 5/6897; A61B 2505/09; A61B 2560/0223; A61B 2562/227; G16H 20/70

(57) ABSTRACT

A system, method and apparatus for diagnosis and therapy. Preferably, the system, method and apparatus is provided for diagnosis and therapy of neurological and/or neuromuscular deficits by using a computational device. Optionally and preferably, the system, method and apparatus track one or more physical movements of the user, which are then analyzed to determine whether the user has one or more neurological and/or neuromuscular deficits. Additionally or alternatively, the system, method and apparatus induce the user to perform one or more physical movements, whether to diagnose such one or more neurological and/or neuromuscular deficits, to treat such one or more neurological and/or neuromuscular deficits, or a combination thereof.

### FIELD OF THE INVENTION

The present invention is of a system, method and apparatus for diagnosis and therapy, and in particular, to such a system, method and apparatus for diagnosis and therapy of neurological and/or neuromuscular deficits.

### BACKGROUND OF THE INVENTION

Patients who suffer from one or more neurological and/or neuromuscular deficits often need specialized therapy in order to regain at least partial functionality, for example in terms of ADL (activities of daily living). For example, specialized physical therapy may be required to enable a patient suffering from a brain injury, such as a stroke or traumatic brain injury, to regain at least some lost functionality. However such specialized physical therapy requires dedicated, highly trained therapists, and so may not be available to all patients who need it.

Although various games and other solutions are available for physical therapy, none of them are designed for the specific needs of patients having neuromuscular or neurological deficits. Such patients require solutions that feature a much more granular and calibrated ability to isolate specific body parts and encourage a simulated range of motions that influence the virtual capabilities of the patient. Such an ability would have a significant impact on accelerating, extending and broadening patient recovery, while at the same time providing important psychological motivation and support.

This is especially important within the first few weeks following a trauma when the neuroadaptive and neuroplastic capacities of the patient are most likely to benefit from additional motivational treatment. However, for these patients in particular, any solution has many stringent requirements which are not currently being met. For example, such patients require personalized treatments that are based on an understanding of the pathologies involved and a variety of therapeutic techniques for treating them. On the other hand, gaming or other physical activities for such patients should not require the use of any tools (e.g, joysticks), as the patients may not be able to use them. Any solution should have graduated levels of difficulty that are based on an integrated understanding of brain sciences, neuroplasticity and self-motivated learning, which can also be personalized for each patient. Unfortunately, no such solution is currently available.

### BRIEF SUMMARY OF THE INVENTION

The present invention provides, in at least some embodiments, a system, method and apparatus for diagnosis and therapy. Preferably, the system, method and apparatus is provided for diagnosis and therapy of neurological and/or neuromuscular deficits by using a computational device. Optionally and preferably, the system, method and apparatus track one or more physical movements of the user, which are then analyzed to determine whether the user has one or more neurological and/or neuromuscular deficits. Additionally or alternatively, the system, method and apparatus monitor the user performing one or more physical movements, whether to diagnose such one or more neurological and/or neuromuscular deficits, to treat such one or more neurological and/or neuromuscular deficits, or a combination thereof.

By “neurological deficit”, it is meant any type of central nervous system deficit, peripheral nervous system deficit, or combination thereof, whether due to injury, disease or a combination thereof. Non-limiting examples of causes for such deficits include stroke and traumatic brain injury.

By “neuromuscular deficit” it is meant any combination of any type of neurological deficit with a muscular component, or any deficit that has both a neurological deficit and a muscular deficit, or optionally any deficit that is musculoskeletal in origin.

In regard to a physical user limitation, such as a limited range of motion in at least one body part (for example, a limited range of motion when lifting an arm), the “limitation” is preferably determined according to the normal or expected physical action or activity that the user would have been expected to engage in, without the presence of the limitation.

A physical limitation or deficit may optionally have a neurological or neuromuscular cause, but is referred to herein generally as a “physical” limitation deficit in regard to the impact that it has on movement of one or more body parts of the user.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computer” on a “computer network”, it should be noted that optionally any device featuring a data processor and the ability to execute one or more instructions may be described as a computer or as a computational device, including but not limited to any type of personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, or a pager. Any two or more of such devices in communication with each other may optionally comprise a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1A shows an exemplary, illustrative non-limiting system according to at least some embodiments of the present invention;

FIG. 1B shows an exemplary, illustrative non-limiting method for calibration according to at least some embodiments of the present invention;

FIG. 2 shows an exemplary, illustrative non-limiting game layer according to at least some embodiments of the present invention;

FIG. 3 shows another exemplary, illustrative non-limiting system according to at least some embodiments of the present invention;

FIG. 4 shows an exemplary, illustrative non-limiting flow for providing tracking feedback according to at least some embodiments of the present invention;

FIG. 5 shows an exemplary, illustrative non-limiting flow for providing tracking according to at least some embodiments of the present invention;

FIG. 6 shows an exemplary, illustrative non-limiting flow for gesture providers according to at least some embodiments of the present invention;

FIG. 7 shows an exemplary, illustrative non-limiting flow for gesture calibration according to at least some embodiments of the present invention;

FIG. 8 shows an exemplary, illustrative non-limiting flow for game flow according to at least some embodiments of the present invention;

FIG. 9 shows an exemplary, illustrative non-limiting flow for providing core functions according to at least some embodiments of the present invention;

FIGS. 10A and 10B show an exemplary, illustrative non-limiting flow for the user interface (UI) according to at least some embodiments of the present invention;

FIG. 11A shows an exemplary, illustrative non-limiting flow for providing license functions according to at least some embodiments of the present invention;

FIG. 11B shows an exemplary, illustrative non-limiting method for privacy protection according to at least some embodiments of the present invention;

FIGS. 12A and 12B relate to an exemplary, illustrative, non-limiting architecture for a system launcher according to at least some embodiments of the present invention;

FIG. 13 shows an exemplary, illustrative, non-limiting architecture for a user interface according to at least some embodiments of the present invention;

FIG. 14 shows an exemplary, illustrative, non-limiting architecture for a user server according to at least some embodiments of the present invention;

FIG. 15 shows an exemplary, illustrative, non-limiting input flow for an exemplary game according to at least some embodiments of the present invention;

FIGS. 16A-16C show an exemplary, illustrative, non-limiting session flow according to at least some embodiments of the present invention;

FIGS. 17A-17D show another exemplary, illustrative, non-limiting session flow according to at least some embodiments of the present invention; and

FIGS. 18A and 18B show exemplary, non-limiting screenshots of example games according to at least some embodiments of the present invention.

### DESCRIPTION OF AT LEAST SOME EMBODIMENTS

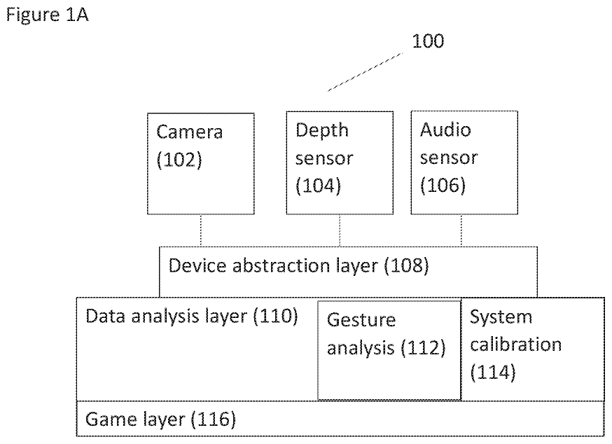

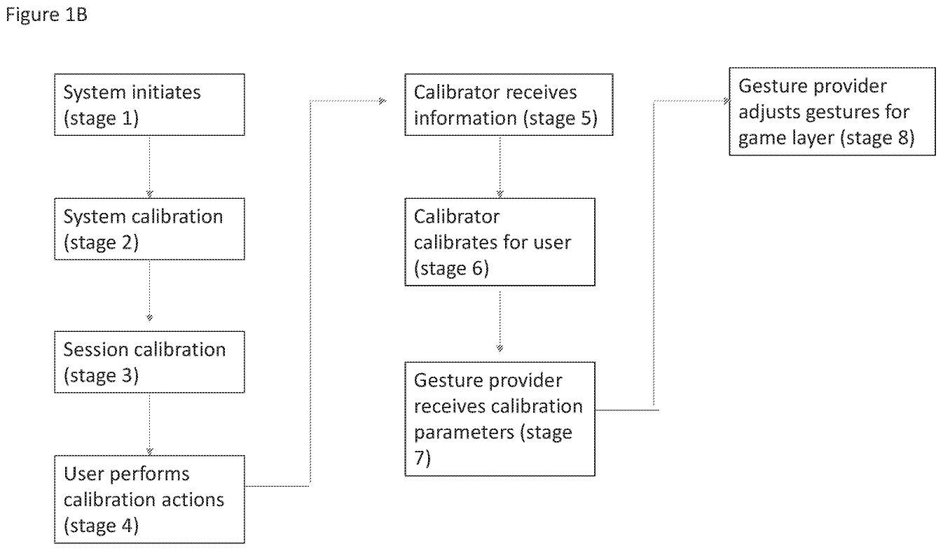

FIG. 1A shows an exemplary, illustrative non-limiting system according to at least some embodiments of the present invention. As shown, a system 100 features a camera 102, a depth sensor 104 and optionally an audio sensor 106. As described in greater detail below, optionally camera 102 and depth sensor 104 are combined in a single product, such as the Kinect product of Microsoft, and/or as described with regard to U.S. Pat. No. 8,379,101, for example. Optionally all three sensors are combined in a single product. The sensor data preferably relates to the physical actions of a user (not shown), which are accessible to the sensors. For example, camera 102 may optionally collect video data of one or more movements of the user, while depth sensor 104 may optionally provide data to determine the three dimensional location of the user in space according to the distance from depth sensor 104. Depth sensor 104 preferably provides TOF (time of flight) data regarding the position of the user; the combination with video data from camera 102 allows a three dimensional map of the user in the environment to be determined. As described in greater detail below, such a map enables the physical actions of the user to be accurately determined, for example with regard to gestures made by the user. Audio sensor 106 preferably collects audio data regarding any sounds made by the user, optionally including but not limited to, speech.

Sensor data from the sensors is collected by a device abstraction layer 108, which preferably converts the sensor signals into data which is sensor-agnostic. Device abstraction layer 108 preferably handles all of the necessary preprocessing such that if different sensors are substituted, only changes to device abstraction layer 108 would be required; the remainder of system 100 would preferably continuing functioning without changes, or at least without substantive changes. Device abstraction layer 108 preferably also cleans up the signals, for example to remove or at least reduce noise as necessary, and may optionally also normalize the signals. Device abstraction layer 108 may be operated by a computational device (not shown). Any method steps performed herein may optionally be performed by a computational device; also all modules and interfaces shown herein are assumed to incorporate, or to be operated by, a computational device, even if not shown.

The preprocessed signal data from the sensors is then passed to a data analysis layer 110, which preferably performs data analysis on the sensor data for consumption by a game layer 116. By “game” it is optionally meant any type of interaction with a user. Preferably such analysis includes gesture analysis, performed by a gesture analysis module 112. Gesture analysis module 112 preferably decomposes physical actions made by the user to a series of gestures. A “gesture” in this case may optionally include an action taken by a plurality of body parts of the user, such as taking a step while swinging an arm, lifting an arm while bending forward, moving both arms and so forth. The series of gestures is then provided to game layer 116, which translates these gestures into game play actions. For example and without limitation, and as described in greater detail below, a physical action taken by the user to lift an arm is a gesture which could translate in the game as lifting a virtual game object.

Data analysis layer 110 also preferably includes a system calibration module 114. As described in greater detail below, system calibration module 114 optionally and preferably calibrates the physical action(s) of the user before game play starts. For example, if a user has a limited range of motion in one arm, in comparison to a normal or typical subject, this limited range of motion is preferably determined as being the user's full range of motion for that arm before game play begins. When playing the game, data analysis layer 110 may indicate to game layer 116 that the user has engaged the full range of motion in that arm according to the user calibration—even if the user's full range of motion exhibits a limitation. As described in greater detail below, preferably each gesture is calibrated separately.

System calibration module 114 may optionally perform calibration of the sensors in regard to the requirements of game play; however, preferably device abstraction layer 108 performs any sensor specific calibration. Optionally the sensors may be packaged in a device, such as the Kinect, which performs its own sensor specific calibration.

FIG. 1B shows an exemplary, illustrative non-limiting method for calibration according to at least some embodiments of the present invention. As shown, in stage 1, the system initiates function. The system may optionally be implemented as described in FIG. 1A but may also optionally be implemented in other ways, for example as described herein. In stage 2, the system performs system calibration, which may optionally include determining license and/or privacy features as described in greater detail below. System calibration may also optionally include calibration of one or more functions of a sensor as described in greater detail herein.

In stage 3, session calibration is optionally performed. By “session”, it is meant the interactions of a particular user with the system. Session calibration may optionally include determining whether the user is placed correctly in regard to the sensors, such as whether the user is placed correctly in regard to the camera and depth sensor. As described in greater detail below, if the user is not placed correctly, the system may optionally cause a message to be displayed to user, preferably at least in a visual display and/or audio display, but optionally in a combination thereof. The message indicates to the user that the user needs to adjust his or her placement relative to one or more sensors. For example, the user may need to adjust his or her placement relative to the camera and/or depth sensor. Such placement may optionally include adjusting the location of a specific body part, such as of the arm and/or hand of the user.

Optionally and preferably, at least the type of game that the user will engage in is indicated as part of the session calibration. For example, the type of game may require the user to be standing, or may permit the user to be standing, sitting, or even lying down. The type of game may optionally engage the body of the user or may alternatively engage specific body part(s), such as the shoulder, hand and arm for example. Such information is preferably provided so that the correct or optimal user position may be determined for the type of game(s) to be played. If more than one type of game is to be played, optionally this calibration is repeated for each type of game or alternatively may only be performed once.

Alternatively, the calibration process may optionally be sufficiently broad such that the type of game does not need to be predetermined. In this non-limiting example, the user could potentially play a plurality of games or even all of the games, according to one calibration process. If the user is potentially not physically capable of performing one or more actions as required, for example by being able to remain standing, and hence could not play one or more games, optionally a therapist who is controlling the system could decide on which game(s) could be played.

In stage 4, user calibration is performed, to determine whether the user has any physical limitations. User calibration is preferably adjusted according to the type of game to be played as noted above. For example, for a game requiring the user to take a step, user calibration is preferably performed to determine whether the user has any physical limitations when taking a step. Alternatively, for a game requiring the user to lift his or her arm, user calibration is preferably performed to determine whether the user has any physical limitations when lifting his or her arm. If game play is to focus on one side of the body, then user calibration preferably includes determining whether the user has any limitations for one or more body parts on that side of the body.

User calibration is preferably performed separately for each gesture required in a game. For example, if a game requires the user to both lift an arm and a leg, preferably each such gesture is calibrated separately for the user, to determine any user limitations. As noted above, user calibration for each gesture is used to inform the game layer of what can be considered a full range of motion for that gesture for that specific user.

In stage 5, such calibration information is received by a calibrator, such as the previously described system calibration module for example. In stage 6, the calibrator preferably compares the actions taken by the user to an expected full range of motion action, and then determines whether the user has any limitations. These limitations are then preferably modeled separately for each gesture.

In stage 7, the gesture provider receives calibration parameters. In stage 8, the gesture provider adjusts gestures according to the modeled limitations for the game layer, as described in greater detail below. The gesture provider therefore preferably abstracts the calibration and the modeled limitations, such that the game layer relates only to the determination of the expected full range of motion for a particular gesture by the user. However, the gesture provider may also optionally represent the deficit(s) of a particular user to the game layer (not shown), such that the system may optionally recommend a particular game or games, or type of game or games, for the user to play, in order to provide a diagnostic and/or therapeutic effect for the user according to the specific deficit(s) of that user.

The system according to at least some embodiments of the present invention preferably monitors a user behavior. The behavior is optionally selected from the group consisting of a performing physical action, response time for performing the physical action and accuracy in performing the physical action. Optionally, the physical action comprises a physical movement of at least one body part. The system is optionally further adapted for therapy and/or diagnosis of a user behavior.

Optionally, alternatively or additionally, the system according to at least some embodiments is adapted for cognitive therapy of the user through an interactive computer program. For example, the system is optionally adapted for performing an exercise for cognitive training.

Optionally the exercise for cognitive training is selected from the group consisting of attention, memory, and executive function.

Optionally the system calibration module further determines if the user has a cognitive deficit, such that the system calibration module also calibrates for the cognitive deficit if present.

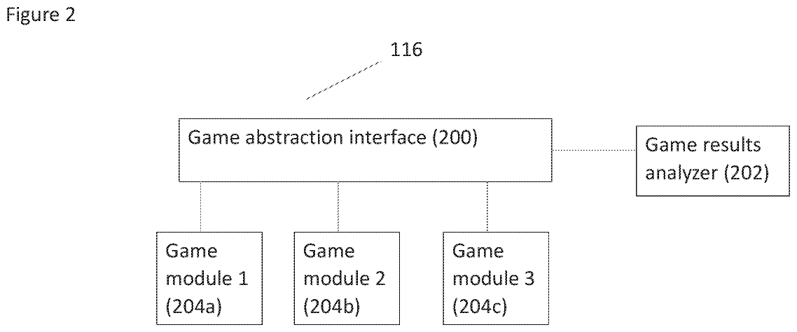

FIG. 2 shows an exemplary, illustrative non-limiting game layer according to at least some embodiments of the present invention. The game layer shown in FIG. 2 may optionally be implemented for the game layer of FIG. 1A and hence is labeled as game layer 116; however, alternatively the game layer of FIG. 1A may optionally be implemented in different ways.

As shown, game layer 116 preferably features a game abstraction interface 200. Game abstraction interface 200 preferably provides an abstract representation of the gesture information to a plurality of game modules 204, of which only three are shown for the purpose of description only and without any intention of being limiting. The abstraction of the gesture information by game abstraction interface 200 means that changes to data analysis layer 110, for example in terms of gesture analysis and representation by gesture analysis module 112, may optionally only require changes to game abstraction interface 200 and not to game modules 204. Game abstraction interface 200 preferably provides an abstraction of the gesture information and also optionally and preferably what the gesture information represents, in terms of one or more user deficits. In terms of one or more user deficits, game abstraction interface 200 may optionally poll game modules 204, to determine which game module(s) 204 would be most appropriate for that user. Alternatively or additionally, game abstraction interface 200 may optionally feature an internal map of the capabilities of each game module 204, and optionally of the different types of game play provided by each game module 204, such that game abstraction interface 200 may optionally be able to recommend one or more games to the user according to an estimation of any user deficits determined by the previously described calibration process. Of course, such information could also optionally be manually entered and/or the game could be manually selected for the user by medical, nursing or therapeutic personnel.

Upon selection of a particular game for the user to play, a particular game module 204 is activated and begins to receive gesture information, optionally according to the previously described calibration process, such that game play can start.

Game abstraction interface 200 also optionally is in communication with a game results analyzer 202. Game results analyzer 202 optionally and preferably analyzes the user behavior and capabilities according to information received back from game module 204 through to game abstraction interface 200. For example, game results analyzer 202 may optionally score the user, as a way to encourage the user to play the game. Also game results analyzer 202 may optionally determine any improvements in user capabilities over time and even in user behavior. An example of the latter may occur when the user is not expending sufficient effort to achieve a therapeutic effect with other therapeutic modalities, but may show improved behavior with a game in terms of expended effort. Of course, increased expended effort is likely to lead to increased improvements in user capabilities, such that improved user behavior may optionally be considered as a sign of potential improvement in user capabilities. Detecting and analyzing such improvements may also optionally be used to determine where to direct medical resources, within the patient population and also for specific patients.

Game layer 116 may optionally comprise any type of application, not just a game. Optionally game results analyzer 202 may optionally analyze the results for the interaction of the user with any type of application.

Game results analyzer 202 may optionally store these results locally or alternatively, or additionally, may optionally transmit these results to another computational device or system (not shown). Optionally, the results feature anonymous data, for example to improve game play but without any information that ties the results to the game playing user's identity or any user parameters.

Also optionally, the results feature anonymized data, in which an exact identifier for the game playing user, such as the user's name and/or national identity number, is not kept; but some information about the game playing user is retained, including but not limited to one or more of age, disease, capacity limitation, diagnosis, gender, time of first diagnosis and so forth. Optionally such anonymized data is only retained upon particular request of a user controlling the system, such as a therapist for example, in order to permit data analysis to help suggest better therapy for the game playing user, for example, and/or to help diagnose the game playing user (or to adjust that diagnosis).

Optionally the following information is transmitted and/or other analyzed, at least to improve game play:
Game results (generated after each game)
   Game Id
   User Id
   Date
   Score
   Duration
   Level (of difficulty)
   Active side (left/right/both)
Calibration results (generated after calibration)
   Calibrator Id
   Date
   Information relative to the calibration (e.g. elevation angle or max forearm pronation angle)
Usage statistics
   Number of software launches
   Login time per user
   In game time per user
System stability
   Log, errors, warnings
UI (user interface) behavior
   Number of click action per button
   Time spend into each part of the software menus
Tracking
   Overall tracking quality (confidence indicator)
   Time with low quality tracking (confidence value is under a certain threshold during games)

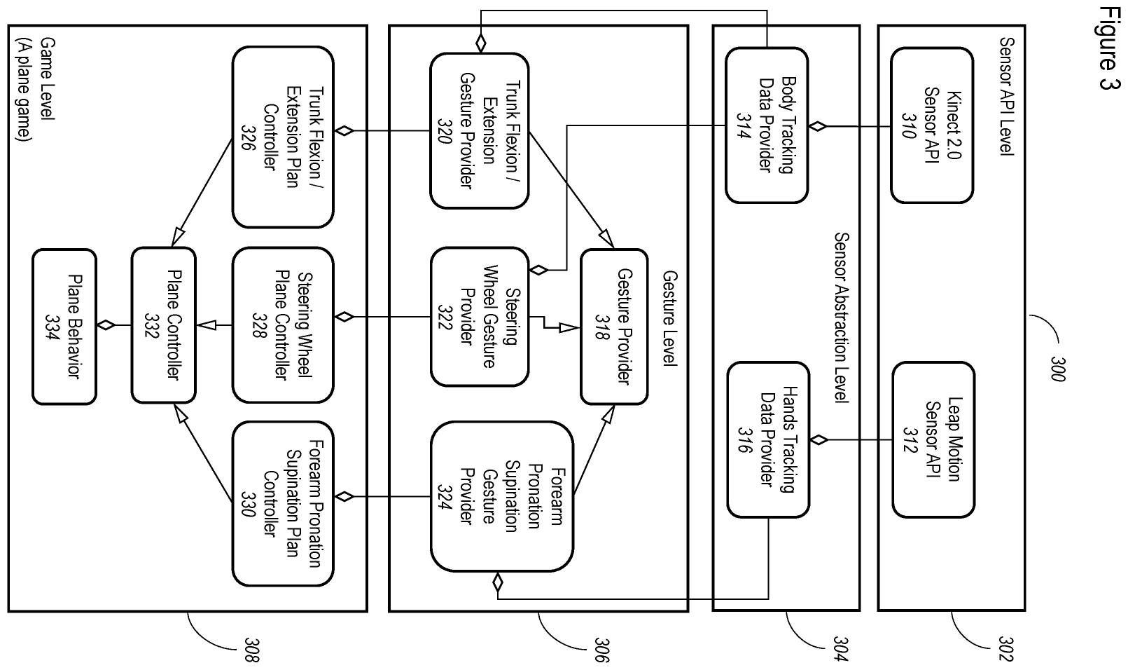

FIG. 3 shows another exemplary, illustrative non-limiting system according to at least some embodiments of the present invention. A system 300 may optionally be implemented to include at least some of the features of the system of FIG. 1; also aspects of system 300 may optionally be swapped with aspects of the system of FIG. 1, and vice versa, such that various combinations, sub-combinations and permutations are possible.

System 300 as shown optionally and preferably includes four levels: a sensor API level 302, a sensor abstraction level 304, a gesture level 306 and a game level 308. Sensor API level 302 preferably communicates with a plurality of sensors (not shown) to receive sensor data from them. According to the non-limiting implementation described herein, the sensors include a Kinect sensor and a Leap Motion sensor, such that sensor API level 302 as shown includes a Kinect sensor API 310 and a Leap Motion sensor 312, for receiving sensor data from these sensors. Typically such APIs are third party libraries which are made available by the manufacturer of a particular sensor.

The sensor data is then passed to sensor abstraction level 304, which preferably handles any sensor specific data analysis or processing, such that the remaining components of system 300 can be at least somewhat sensor agnostic. Furthermore, changes to the sensors themselves preferably only necessitate changes to sensor API level 302 and optionally also to sensor abstraction level 304, but preferably not to other levels of system 300.

Sensor abstraction level 304 preferably features a body tracking data provider 314 and a hands tracking data provider 316. Optionally all parts of the body could be tracked with a single tracking data provider, or additional or different body parts could optionally be tracked (not shown). For this implementation, with the two sensors shown, preferably data from the Kinect sensor is tracked by body tracking data provider 314, while data from the Leap Motion sensor is tracked by hands tracking data provider 316.

Next, the tracked body and hand data is provided to gesture level 306, which includes modules featuring the functionality of a gesture provider 318, from which specific classes inherit their functionality as described in greater detail below. Gesture level 306 also preferably includes a plurality of specific gesture providers, of which only three are shown for the purpose of illustration only and without any intention of being limiting. The specific gesture providers preferably include a trunk flexion/extension gesture provider 320, which provides information regarding leaning of the trunk; a steering wheel gesture provider 322, which provides information regarding the user interactions with a virtual steering wheel that the user could grab with his/her hands; and a forearm pronation/supination gesture provider 324, which provides information about the rotation of the hand along the arm.

Each gesture provider relates to one specific action which can be translated into game play. As shown, some gesture providers receive information from more than one tracking data provider, while each tracking data provider can feed data into a plurality of gesture providers, which then focus on analyzing and modeling a specific gesture.

A non-limiting list of gesture providers is given below:
ArmsPaddlingGestureProvider—which relates to a paddling motion with the arms, as for example when the user is manipulating a virtual oar and/or is controlling a virtual boat.
ArmsPumpingGestureProvider—which relates to pumping the arm in a single direction, by having the user extend his or her arm in front at the shoulder level.
BodyWeightTransferGestureProvider—which relates to transfer of body weight from one leg to the other.
Steering WheelGestureProvider—as described above, provides information regarding the user interactions with a virtual steering wheel that the user could grab with his/her hands.
FingersFlexionExtensionGestureProvider—relates to closing and opening each finger individually.
FingersPinchGestureProvider—relates to manipulation of at least two specific fingers so that for example they are touching each other, such as for example touching a finger to the thumb.
FootStepGestureProvider—relates to taking a step by the user in terms of activity of each foot.
GestureProvider—this is the generic provider format from which other providers may be determined.
HandGrabbingGestureProvider—relates to reaching out to grasp and optionally manipulate an object with a hand, including opening and closing the hand (in this non-limiting example, only provided with Leap Motion data; the actual opening and closing of the fingers is handled separately).
HandPronationSupinationGestureProvider—provides information about the rotation of the hand (same as forearm pronation/supination).
HandsObjectsGraspingGestureProvider—only to move hand to reach a virtual object and then move it; remaining with hand at the virtual object for a predetermined period of time is considered to be equivalent to grasping the object (in this non-limiting example, only provided with the Kinect data)
HandsUpGestureProvider—provides information about the raising up a hand.
KneesFlexionExtensionGestureProvider—provides information about bending the knee.
ShouldersFlexionExtensionGestureProvider—provides information about shoulder flexion (lifting the arm out in front of the body and up overhead) and shoulder extension.
ShouldersHorizontalAbductionAdductionGestureProvider—provides information about abduction and adduction movements involving the shoulders. Adduction is the movement of a body part toward the body's midline, while abduction is the movement away from the midline. In this case, the arm is extended and raised before being moved toward, or away from the mindline.
ShouldersLateralAbductionAdductionGestureProvider—provides information about the above gesture performed laterally.
ShouldersToHandsLateralMovementsGestureProvider
TrunkAxialRotationGestureProvider—provides information about twisting of the trunk.
TrunkForwardLeaningGestureProvider—provides information about the trunk leaning backward or forward.
TrunkLateralLeaningGestureProvider—provides information about the trunk leaning from side to side.
WristFlexionExtensionGestureProvider—provides information about bending of the wrist.
WristRadialUlnarDeviationGestureProvider—provides information about rotating the wrist.

An optional additional or alternative gesture provider is a ClimbingGestureProvider, which provides information about a hand-over-hand gesture by the user.

Optionally any of the above gesture provides may be included or not included in regard to a particular game or the system.

Optionally each such gesture provider has a separate calibrator that can calibrate the potential range of motion for a particular user and/or also determine any physical deficits that the user may have in regard to a normal or expected range of motion, as previously described. The gesture providers transform tracking data into normalized output values that will be used by the game controllers of game level 308 as inputs, as described in greater detail below. Those output values are generated by using predefined set of ranges or limits that can be adjusted. For instance, the above Forearm Pronation/Supination Gesture Provider will return a value between −1.0 (pronation) and 1.0 (supination) which represents the current rotation of the forearm along its axis (normalized). Note that initial position (value equals to 0) is defined with the thumb up position. Similar ranges could easily be determined by one of ordinary skill in the art for all such gesture providers.

Suppose that a given patient was not able to perform the full range of motion (−45° to) 45° for such a motion. In that case, the Gesture Provider parameters could be adjusted to allow the patient to cover the full range of the normalized value (−1.0 to 1.0). With those adjustments, the patient will therefore be able to fully play the game like everyone else. This adjustment process is called Gesture Provider Calibration and is a non-limiting example of the process described above. It's important to note that preferably nothing has changed in the game logic; the game always expects a normalized value between −1.0 and 1.0, so the adjustment requires no changes to the game logic.

At game level 308, a plurality of game controllers is provided, of which only three are shown for the sake of description only and without wishing to be limited in any way. These game controllers are shown in the context of a game called the “plane game”, in which the user controls the flight of a virtual plane with his/her body part(s). Each such game controller receives the gesture tracking information from a particular gesture provider, such that trunk flexion/extension gesture provider 320 provides tracking information to a trunk flexion/extension plane controller 326. Steering wheel gesture provider 322 provides tracking information to a steering wheel plane controller 328; and forearm pronation/supination gesture provider 324 provides tracking information to a forearm pronation/supination plane controller 330.

Each of these specific game controllers feeds in information to a general plane controller 332, such the game designer can design a game, such as the plane game, to exhibit specific game behaviors as shown as a plane behaviors module 334. General plane controller 332 determines how the tracking from the gesture providers is fed through the specific controllers and is then provided, in a preferably abstracted manner, to plane behaviors module 334. The game designer would then only need to be aware of the requirements of the general game controller and of the game behaviors module, which would increase the ease of designing, testing and changing games according to user behavior.

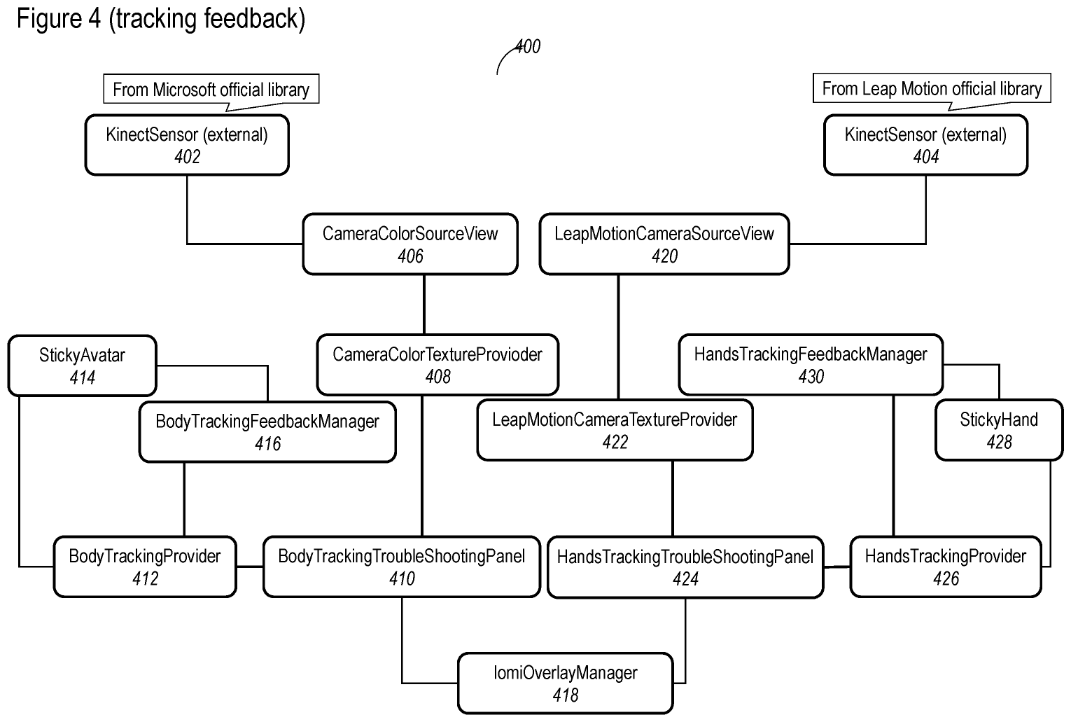

FIG. 4 shows an exemplary, illustrative non-limiting flow for providing tracking feedback according to at least some embodiments of the present invention. A tracking feedback flow 400 preferably includes data from a plurality of sensors, of which APIs for two sensors are shown: a Kinect API 402 and a Leap Motion API 404.

Data from Kinect API 402 first goes to a color camera source view 406, after which the data goes to a camera color texture provider 408. Color camera source view 406 provides raw pixel data from the Kinect camera. Camera color texture provider 408 then translates the raw pixel data to a texture which then can be used for display on the screen, for example for trouble shooting.

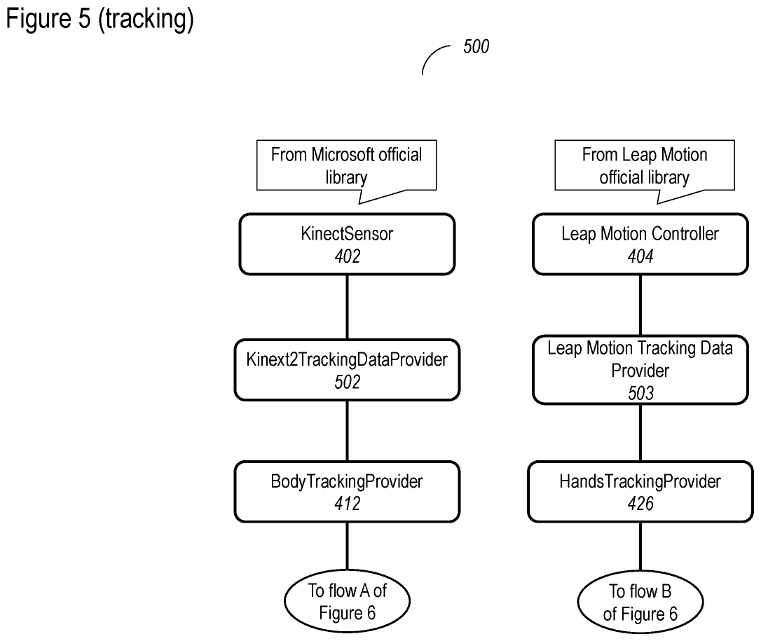

Next the data is provided to an optional body tracking trouble shooting panel 410, which determines for example if the body of the user is in the correct position and optionally also orientation in regard to the Kinect sensor (not shown). From there, the data is provided to a body tracking provider 412, which is also shown in FIG. 5.

For the tracking feedback flow, body tracking provider 412 also preferably communicates with a sticky avatar module 414, which shows an avatar representing the user or a portion of the user, such as the user's hand for example, modeled at least according to the body tracking behavior. Optionally the avatar could also be modeled according to the dimensions or geometry of the user's body. Both sticky avatar module 414 and body tracking provider 412 preferably communicate with a body tracking feedback manager 416. Body tracking feedback manager 416 controls the sticky avatar provided by sticky avatar module 414, which features bones and joints, by translating data from body tracking to visually update the bones and joints. For example, the sticky avatar could optionally be used with this data to provide visual feedback on the user's performance.

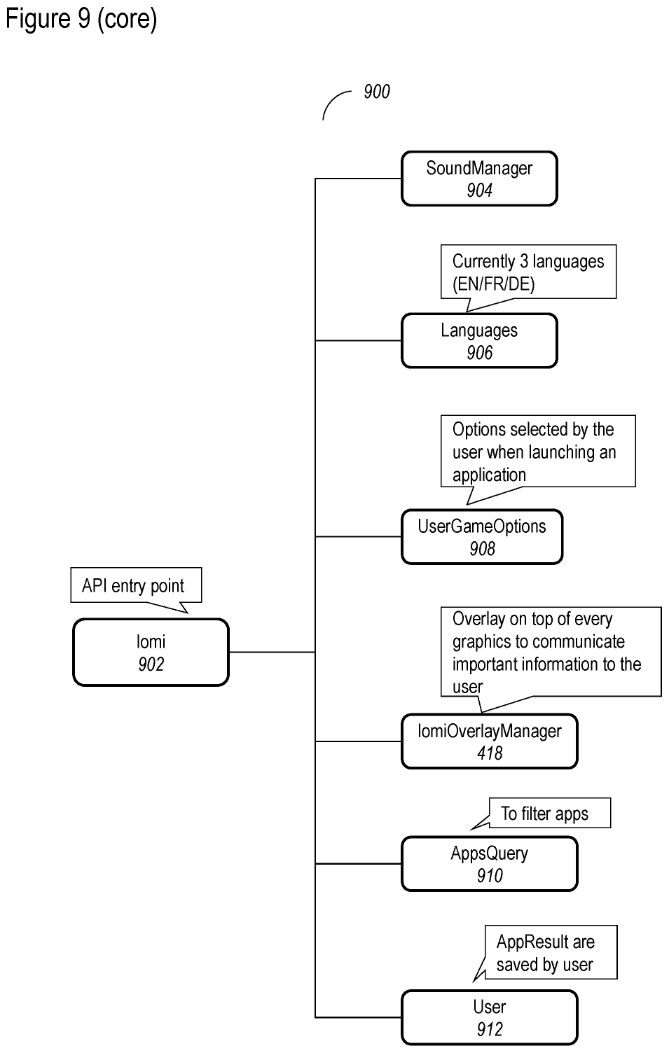

From body tracking trouble shooting panel 410, the data communication preferably moves to an overlay manager 418, which is also shown in FIG. 9. Overlay manager 418 preferably controls the transmission of important messages to the user (which in this case may optionally be the controller of the computational device on which game play is being executed, rather than the user playing the game), which may optionally be provided as an overlay to the user interface. In this non-limiting example, if body tracking trouble shooting panel 410 determines that the body of the user (playing the game) is not correctly positioned with regard to the Kinect sensor, then body tracking trouble shooting panel 410 could provide this information to overlay manager 418. Overlay manager 418 would then cause a message to be displayed to the user controlling the computational device, to indicate the incorrect positioning of the body of the user playing the game.

Turning now to the other side of the drawing, data from Leap Motion API 404 is transmitted to a Leap Motion camera source view 420. Data goes to a Leap Motion camera texture provider 422. Leap Motion camera source view 420 provides raw pixel data from the Leap Motion device. Leap Motion camera texture provider 422 then translates the raw pixel data to a texture which then can be used for display on the screen, for example for trouble shooting.

Next the data is provided to an optional hand tracking trouble shooting panel 424, which determines for example if the hand or hands of the user is/are in the correct position and optionally also orientation in regard to the Leap Motion sensor (not shown). From there, the data is provided to a hand tracking provider 426, which is also shown in FIG. 5.

For the tracking feedback flow, a hand tracking provider 426 also preferably communicates with a sticky hand module 428, which shows an avatar representing the user's hand or hands, modeled at least according to the hand tracking behavior. Optionally the hand could also be modeled according to the dimensions or geometry of the user's hand(s). Both sticky avatar module 414 and hand tracking provider 426 preferably communicate with a hand tracking feedback manager 430.

From hand tracking trouble shooting panel 424, the data communication preferably moves to the previously described overlay manager 418. In this non-limiting example, if hand tracking trouble shooting panel 424 determines that the hand(s) of the user (playing the game) is not correctly positioned with regard to the Leap Motion sensor, then hand tracking trouble shooting panel 424 could provide this information to overlay manager 418. Overlay manager 418 would then cause a message to be displayed to the user controlling the computational device, to indicate the incorrect positioning of the hand(s) of the user playing the game.

FIG. 5 shows an exemplary, illustrative non-limiting flow for providing tracking according to at least some embodiments of the present invention. Some of the same components with the same function are shown as in FIG. 4; these components have the same numbering. As shown, a flow 500 again features two sensor APIs as shown. Kinect API 402 preferably communicates with a Kinect tracking data provider 504. Leap Motion API 404 preferably communicates with a Leap Motion tracking data provider 503. The remaining components are described with regard to FIG. 4.

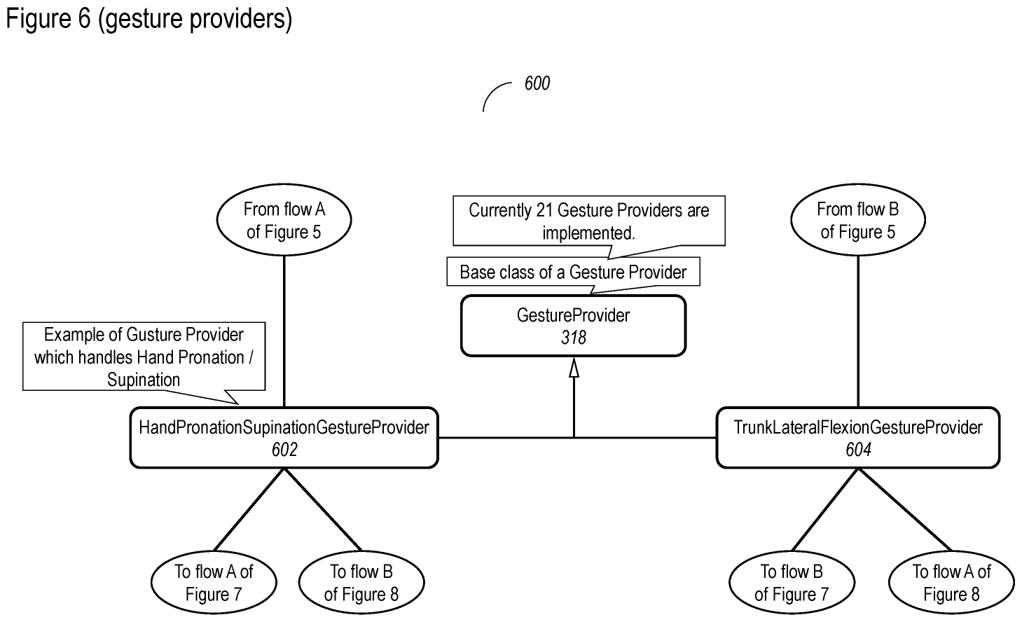

FIG. 6 shows an exemplary, illustrative non-limiting flow for gesture providers according to at least some embodiments of the present invention. Some of the same components with the same function are shown as in FIGS. 4 and 5; these components have the same numbering. As shown with regard to a flow 600, connections are made from the flows A and B of FIG. 5. General gesture provider 318 from FIG. 3 is shown. Non-limiting examples of specific gesture providers are shown as a hand pronation/supination gesture provider 602 and a trunk lateral/flexion gesture provider 604.

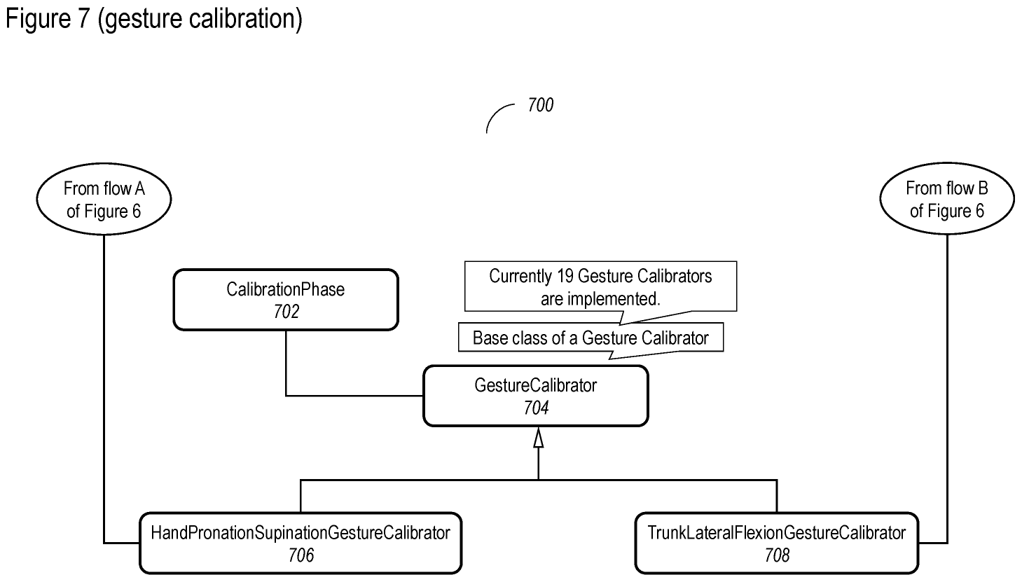

FIG. 7 shows an exemplary, illustrative non-limiting flow for gesture calibration according to at least some embodiments of the present invention. As shown a flow 700 features a calibration phase controller 702, which operates to control the user calibration phase as previously described. Calibration phase controller 702 sends instructions to a gesture calibrator 704, which may optionally operate to perform the user calibration phase as previously described with regard to a plurality of gestures overall. Preferably calibration for each gesture is performed by a separate specific gesture calibrator, of which two non-limiting examples are shown: a hand pronation/supination gesture controller 706 and a trunk lateral flexion gesture calibrator 708.

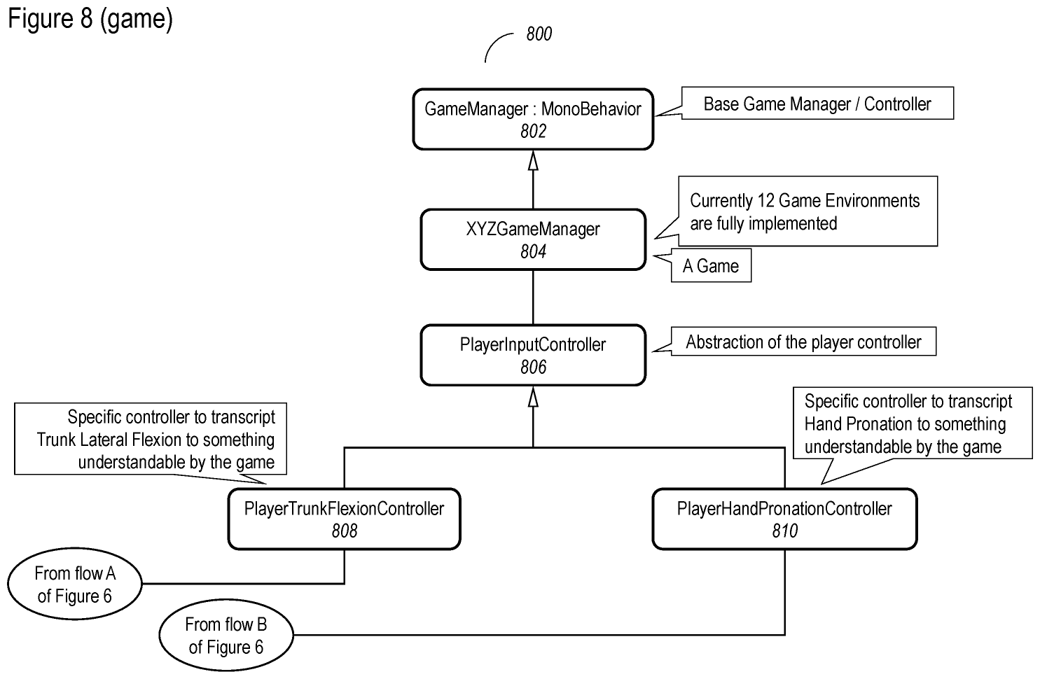

FIG. 8 shows an exemplary, illustrative non-limiting flow for game play according to at least some embodiments of the present invention. As shown, a game play flow 800 features a generic game manager 802 which is in communication with a specific game manager 804, of which only one is shown but of which a plurality may optionally be provided (not shown). Each game manager 804 manages a player input controller 806, through which player input to the game is provided. Player input is preferably provided through the previously described game controllers, of which two are shown for the sake of illustration only. A player trunk flexion controller 808 receives input from trunk lateral flexion gesture provider 604 (not shown, see FIG. 6). A player hand pronation controller 810 receives input from hand pronation/supination gesture provider 602 (not shown, see FIG. 6).

FIG. 9 shows an exemplary, illustrative non-limiting flow for providing core functions according to at least some embodiments of the present invention. As shown, a flow 900 features a user interface entry point 902 for receiving user commands regarding the function of the system (as opposed to game play, although such user commands could also optionally be received through one of the body/body part tracking methods described herein). User interface entry point 902 preferably controls a sound manager 904 for managing the sound display (and optionally also for receiving voice driven input commands); a language controller 906 for controlling the display language of the GUI (graphical user interface); and a user game options interface 908, for receiving the game option choices made by the user when launching a game. User interface entry point 902 preferably also controls the previously described overlay manager 418 (see FIG. 4 for a complete description).

User interface entry point 902 preferably also controls an apps query module 910 to provide a list of all applications according to criteria, for example to filter by functions, body part, what is analyzed and so forth; and a user app storage module 912, optionally for user's own applications, or for metering the number of applications provided in the license.

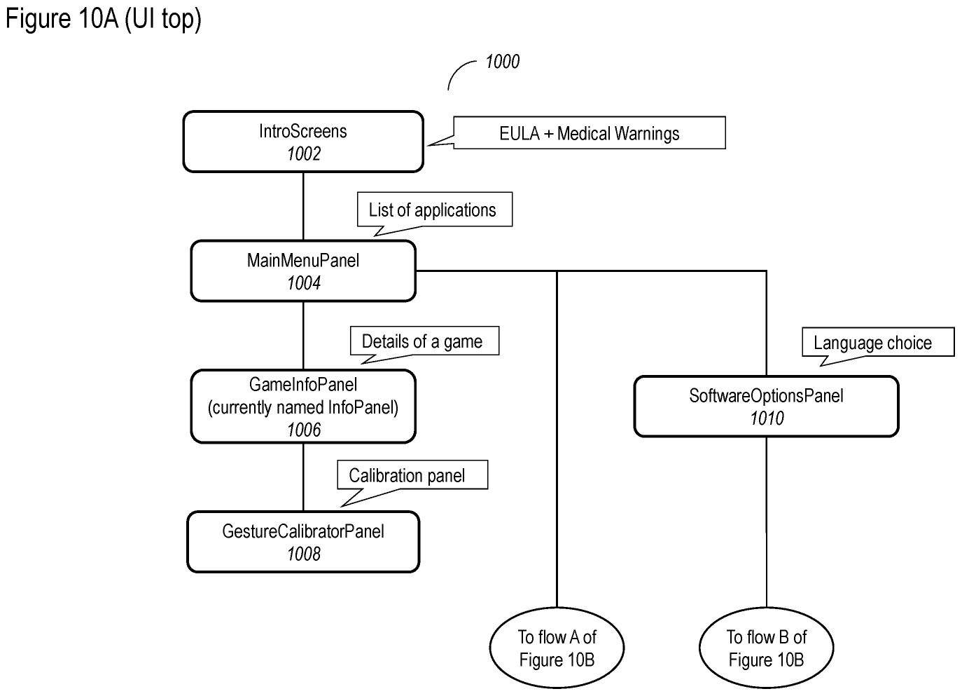

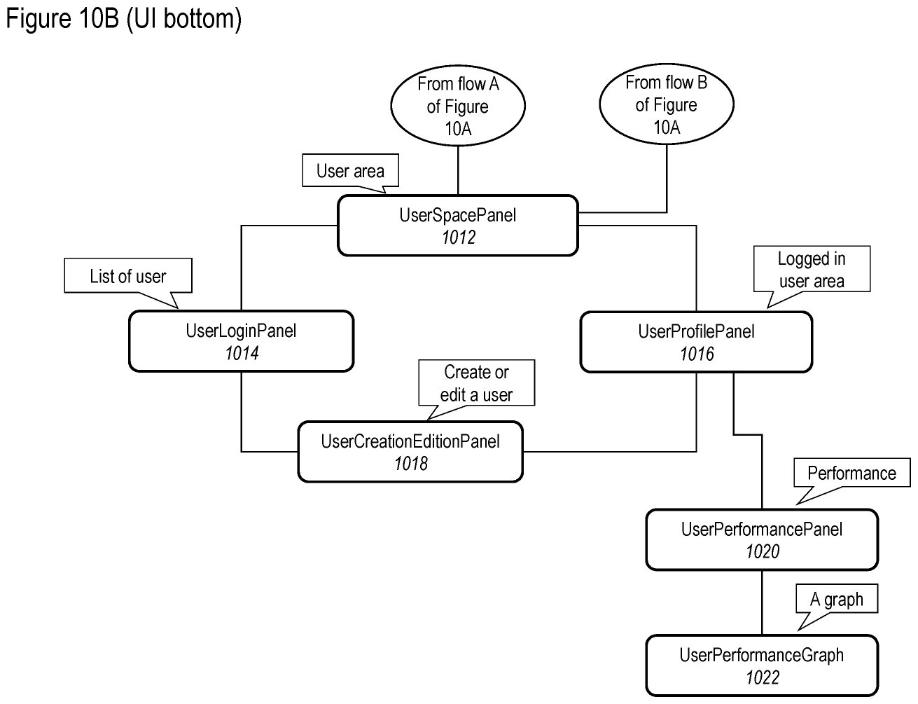

FIGS. 10A and 10B show an exemplary, illustrative non-limiting flow for the user interface (UI) according to at least some embodiments of the present invention, indicating a non-limiting way in which the user may optionally interact with the non-limiting implementation of the system as described herein with regard to FIGS. 3-9. As shown in FIG. 10A, a flow 1000 preferably starts with one or more intro screens 1002, which may optionally include one or more of a EULA or other software license, a privacy warning, a privacy check and so forth.

Next in a main menu panel 1004, the user may optionally be presented with a list of choices to made, for example regarding which game to play and/or which user deficits to be diagnosed or corrected. From there, once a game is selected, the user is taken to a game information panel 1006 and then to a gesture calibration panel 1008, to initiate the previously described gesture calibration process.

Optionally from main menu panel 1004, the user may select one or more languages through an options panel 1010.

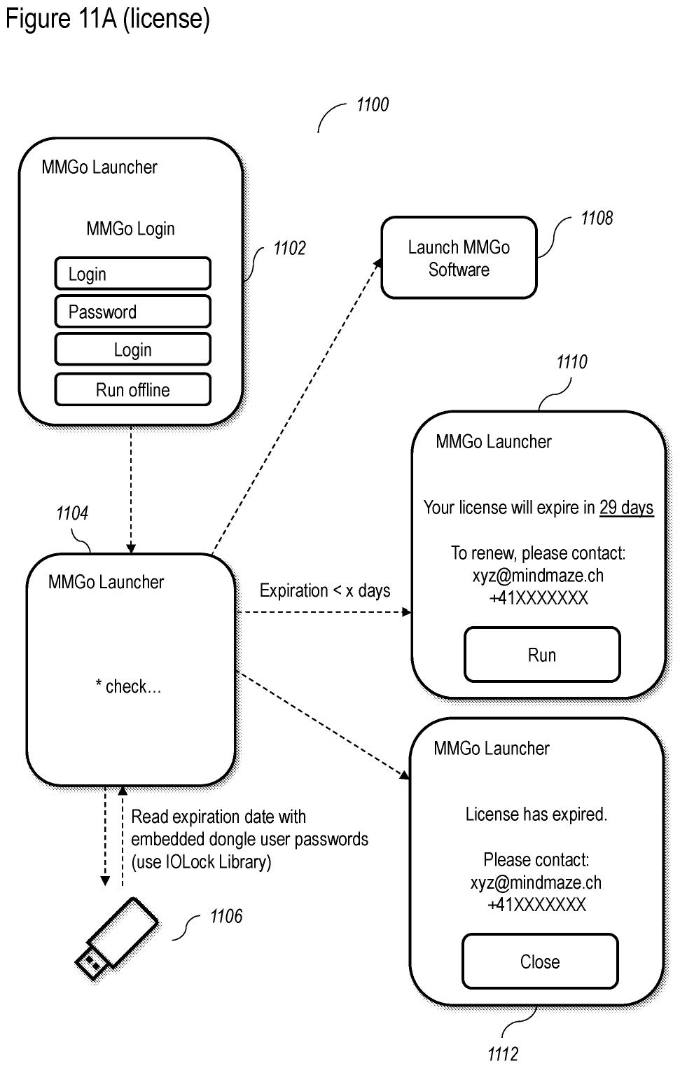

Turning now to FIG. 10B, flow 1000 preferably continues with a user space panel 1012 and then to either a user login panel 1014 or a user profile panel 1016. Optionally, user space panel 1012 provides an interface to all necessary information for the user, and may optionally also act as an overall controller, to decide what the user can see. However, the user has preferably already logged into the system as described in greater detail below with regard to FIG. 11.

The user may then optionally personalize one or more functions in a user creation edition panel 1018.

Next the user optionally can access data regarding a particular user (the “user” in this case is the game player) in a performance panel 1020. This data may optionally be represented as a graph in performance graph 1022.

FIG. 11A shows an exemplary, illustrative non-limiting flow for providing license functions according to at least some embodiments of the present invention. As shown a license function flow 1100 preferably starts with a software launcher 1102 that may optionally provide user login functionality as shown, such as a username and password for example. Other types of login functionality may optionally be provided, additionally or alternatively, including but not limited to a fingerprint scan, a retinal scan, a palmprint scan, a card swipe or a near field communication device.

Next, if the user hasn't done so already, the user is prompted by checking module 1104 to insert a hardware dongle 1106 into a port of the computational device, such as a USB (universal serial bus) port as a non-limiting example. Checking module 1104 checks for user security, optionally to verify user login details match, but at least to verify that dongle 1106 is valid. Checking module 1104 also checks to see if a valid, unexpired license is still available through dongle 1106. If dongle 1106 is not valid or does not contain a license that at one point was valid (even if expired now), the process stops and the software launch is aborted. An error message may optionally be shown.

If dongle 1106 is valid and contains a license that at one point was valid, software launch 1108 continues. Next checking module 1104 checks to see that the license is not expired. If the license is currently valid and not expired, then a time to expiration message 1110 is shown. Otherwise, if the license is expired, then an expired license message 1112 is shown.

FIG. 11B shows an exemplary, illustrative non-limiting method for privacy protection according to at least some embodiments of the present invention. As shown, the method preferably starts with an initiation of the system launch in stage 1. In stage 2, the user logs in. In stage 3, if the dongle or other secondary verification device is not present, the user is asked to present it, for example by inserting it into a port of the computer. In stage 4, the system determines whether the dongle or other secondary verification device is validated. If not, then in stage 5, the system stops, and the user is not able to access any information stored in the system, including without limitation patient details and patient information. The term “patient” in this context refers to users playing a game provided by the system. This provides an additional layer of protection for patient information, as a user who was able to unauthorizedly obtain login details would still not be able to access patient information from the system. Preferably such patient information also cannot be exported from the system without the presence of the dongle or other secondary verification device, again preventing theft of patient information.

In stage 6, access to patient information and other parts of the system are preferably only possible if the dongle or other secondary verification device is validated in stage 4.

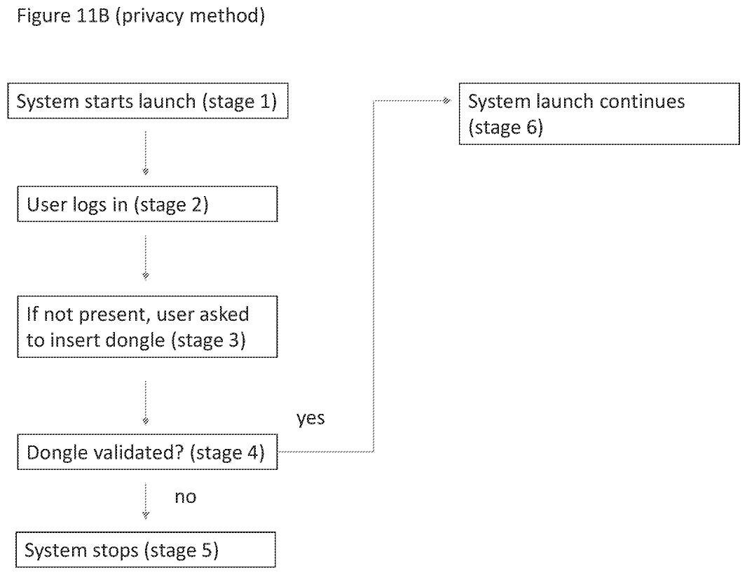

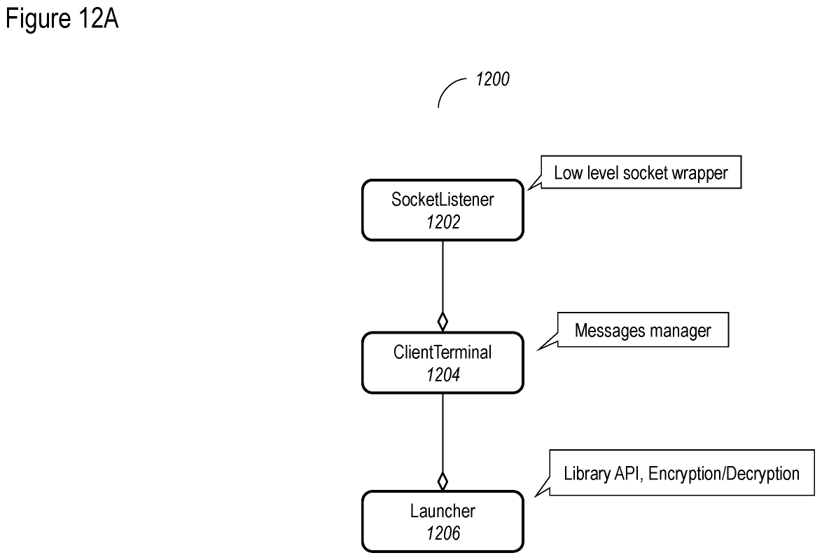

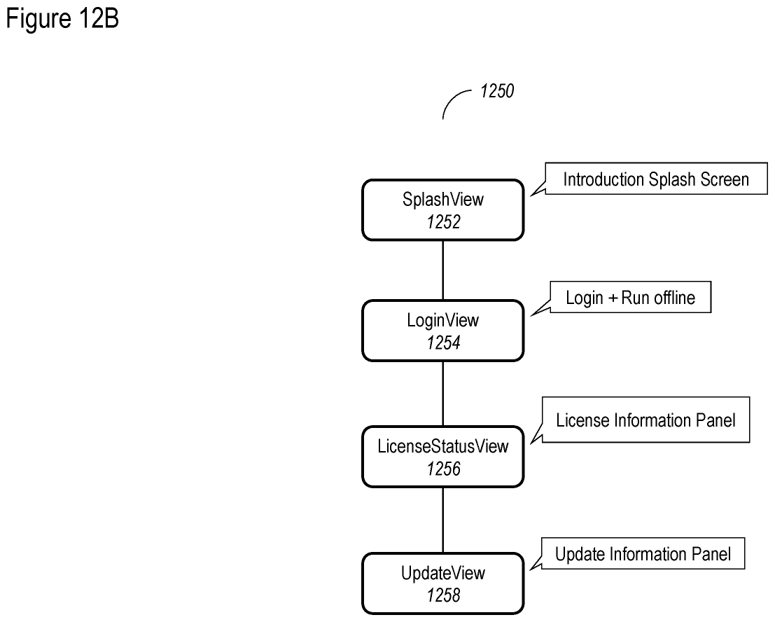

FIGS. 12A and 12B relate to an exemplary, illustrative, non-limiting architecture for a system launcher according to at least some embodiments of the present invention. FIG. 12A relates to an exemplary connector architecture while FIG. 12B relates to an exemplary UI architecture.

A launcher 1200 is initiated upon launch of the system, as shown in FIG. 12A. The launcher is the first screen presented to the user upon starting the previously described system, as shown with regard to UI architecture 1250, shown in FIG. 12B. The system attempts to validate by connecting online, through a connector 1202 of FIG. 12A. A messages manager 1204 then handles the client terminal, followed by a launcher process 1206 to determine whether the system is online. If the system is online, validation is handled through the server.

An initial screen 1252 invites the user to login in a login screen 1254, if the launcher detects that the system is offline or cannot validate through the internet. The Offline validation method optionally includes the time left on the USB Dongle as previously described. Alternatively, launcher 1200 uses the Grace Period (by checking how long the application is allowed to run without license). License status information is preferably provided by a license status view 1256. Any update information is provided by an update view 1258. If a software update is available, preferably update view 1258 enables the user to select and download the software update. Optionally the update is automatically downloaded in the background and then the user is provided with an option as to whether to install. If an update is considered to be urgent or important, optionally it will also install automatically, for example as a default.

When the user successfully logs in, the system is started with the launch of the software interface. From that point, both applications (software interface and launcher) are linked through a TCP channel. If one application dies or loses communication with the other, optionally both die. The Launcher then periodically makes sure the user license is still valid.

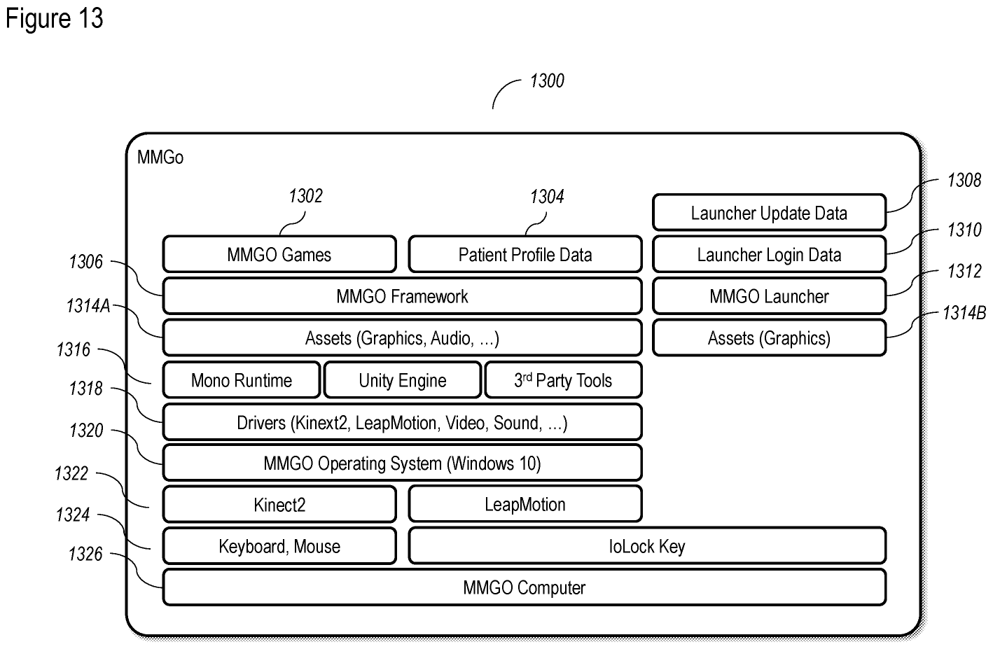

FIG. 13 shows an exemplary, illustrative, non-limiting architecture for a user interface according to at least some embodiments of the present invention. As shown a system 1300 includes a games module 1302 and a patient information module 1304, both of which are specific for a particular session be implemented, in which the session includes one or more specific games being played by a specific patient. On the right hand side, system 1300 includes a launcher sub-system, including a launcher update module 1308 and a launcher login data module 1310. Launcher update module 1308 detects and provides information with regard to software updates as previously described. Launcher login data module 1310 handles authentication and login of the user as previously described.

System 1300 preferably features two core processes for operating a games session and the launcher. The games session is operated by a framework 1306, which supports game play. Launch is operated by a launcher 1312, which optionally operates as described for FIGS. 12A and 12B. Each of framework 1306 and launcher 1312 preferably has access to any necessary assets 1314, shown as assets 1314A for framework 1306 and assets 1314B for launcher 1312.

Framework 1306 is preferably supported by one or more engines 1316, which may optionally be third party engines. For example and without limitation, engines 1316 may optionally include mono runtime, Unity Engine and one or more additional third party engines. Engines 1316 may then optionally be able to communicate with one or more sensors 1322 through one or more drivers 1318. Drivers 1318 in turn communicate with one or more sensors 1322 through an operating system 1320, which assists to abstract data collection and communication. Sensors 1322 may optionally include a Kinect and a Leap Motion sensor, as shown. The user may optionally provide inputs through user inputs 1324, such as a keyboard and mouse for example. All of the various layers are preferably operated by and/or through a computational device 1326 as shown.

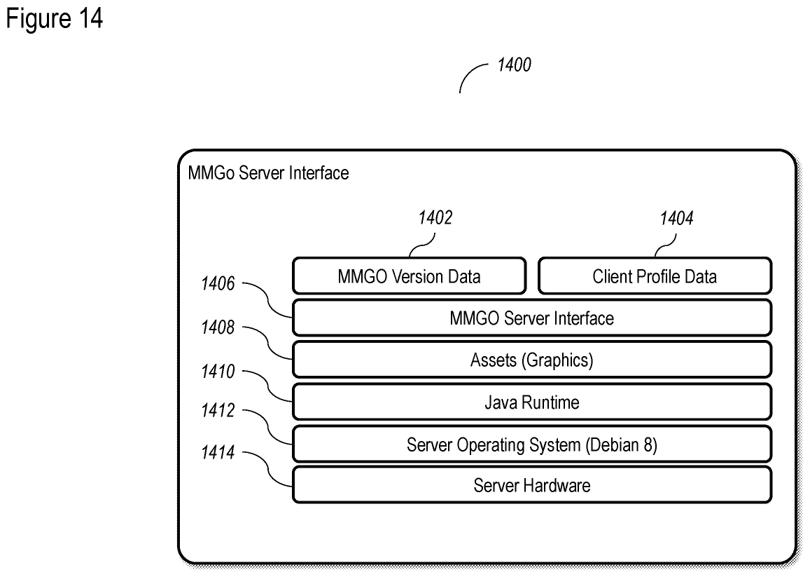

FIG. 14 shows an exemplary, illustrative, non-limiting architecture for a server interface according to at least some embodiments of the present invention. As shown a service interface 1400 receives version data 1402 and client profile data 1404 in order to be able to initiate a session. Profile data 1404 optionally and preferably relates to a specific patient who is interacting with the system for the particular session. A server interface framework 1406 supports interactions between the server and the user computational device. Preferably, server interface framework 1406 receives assets 1408 and operates over a Java runtime engine 1410. Java runtime engine 1410 is operated by a server operating system 1412 over a server 1414.

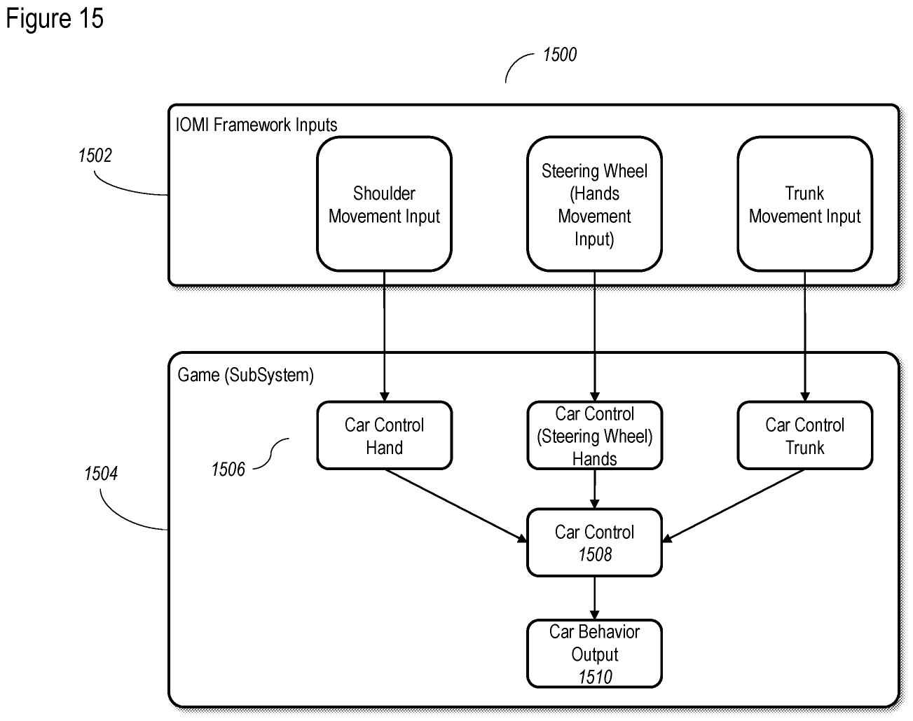

FIG. 15 shows an exemplary, illustrative, non-limiting input flow for an exemplary game according to at least some embodiments of the present invention. As shown, a flow 1500 features inputs from the patient interacting with the system for the session. In this non-limiting example, the game is a car driving game. The patient inputs 1502 include shoulder movement, steering wheel (hand) movement and trunk movement.

Patient inputs 1502 are provided to a car control layer 1506 of a game subsystem 1504. Car control layer 1506 includes control inputs which receive their information directly from patient inputs 1502. For example, a shoulder control input receives information from the shoulder movement of patient inputs 1502. Steering wheel movement from patient inputs 1502 is provided to steering wheel control in car control layer 1506. Trunk movement from patient inputs 1502 is provided to trunk control in car control layer 1506.

Car control layer 1506 then provides the collected inputs to a car control module 1508, to determine how the patient is controlling the car. Car control module 1508 then provides the control information to a car behavior output 1510, which determines how the car in the game will behave, according to the patient movement.

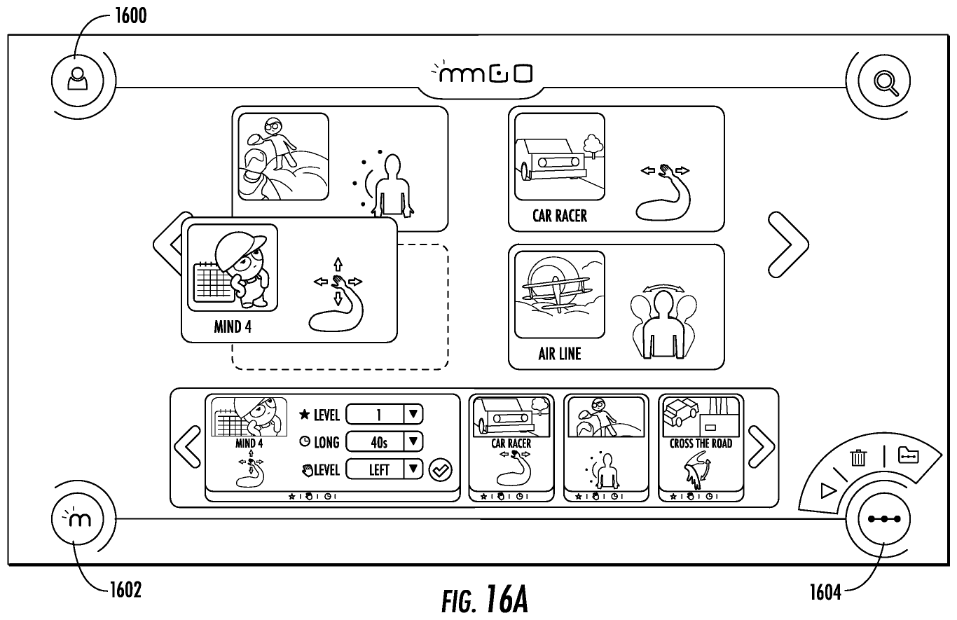

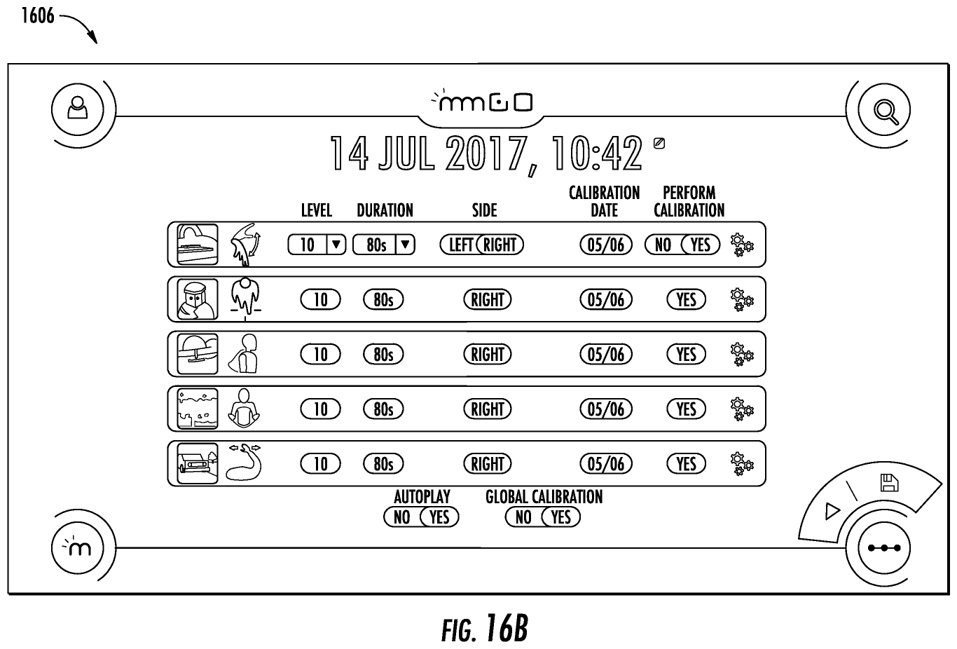

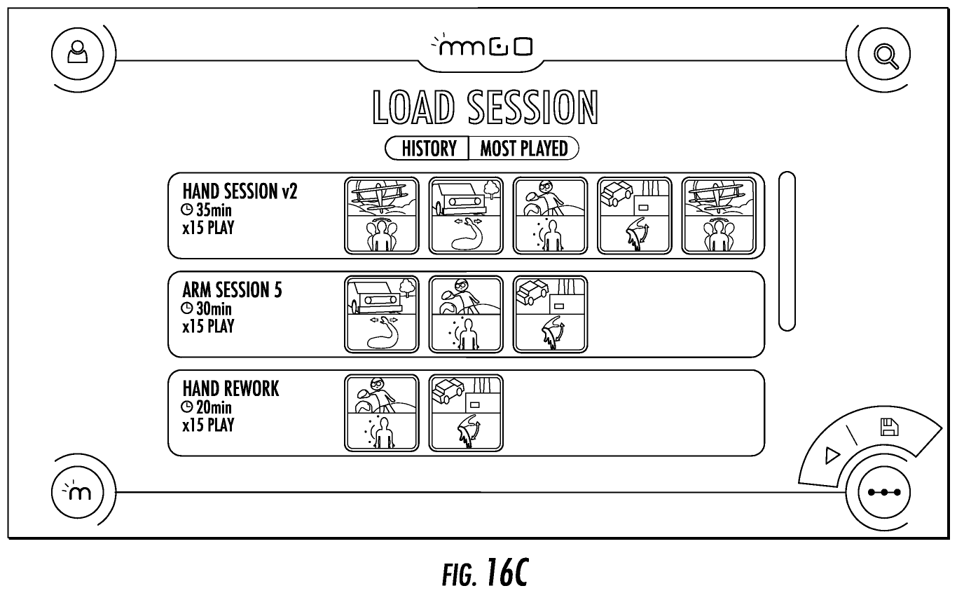

FIGS. 16A-16C show an exemplary, illustrative, non-limiting session flow according to at least some embodiments of the present invention. FIG. 16A shows a session being assembled from a plurality of game action modules. The possible game action modules 1600 are shown on top. The user creating the session may optionally drag and drop one or more game action modules 1600 into a session board 1602, at the bottom. Session board 1602, for each game action module, shows the parameters of the games, such as for example one or more of the level of difficulty, the duration and optionally whether a particular side of the patient is to be emphasized for treatment. The game action modules are shown in order.

A controller 1604 optionally performs one or more of the following: redirect a user to the screen shown in FIG. 16B, to show what would happen during the session; to delete a game module or the session; or to load an existing session or to save the session.

FIG. 16B shows an exemplary completed session that is ready for execution with a patient, with a plurality of game modules in the order in which they will be executed during the session. Optionally, a calibration date is shown, if the particular game module was already calibrated for the patient. The user can optionally play or save the session.

FIG. 16C shows a plurality of sessions, each of which is ready for execution with the patient. The user may optionally choose which session to execute with the patient. All of saved and previously played sessions are shown. The user can either play one session or reuse one and edit it to adapt it to the patient's needs.

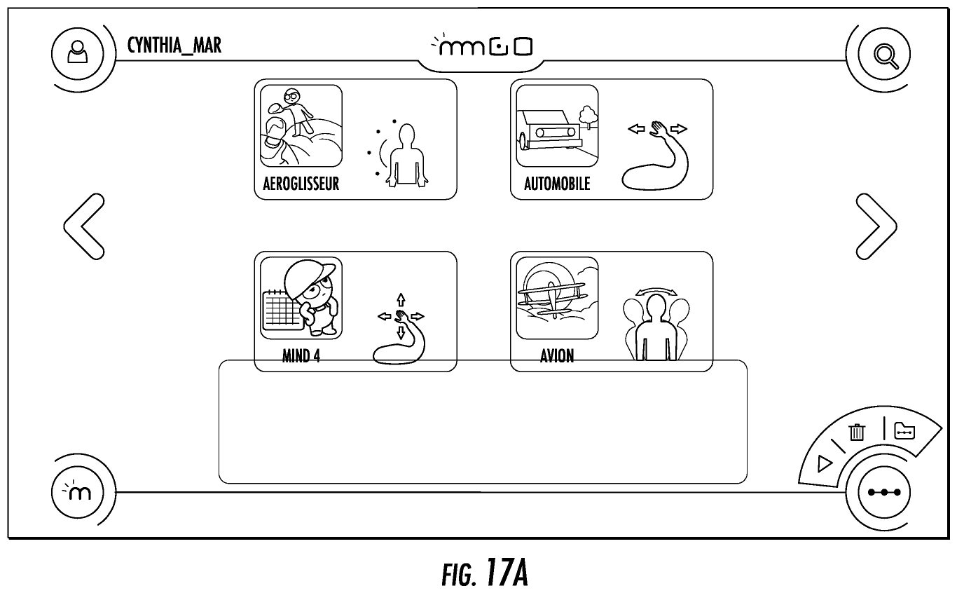

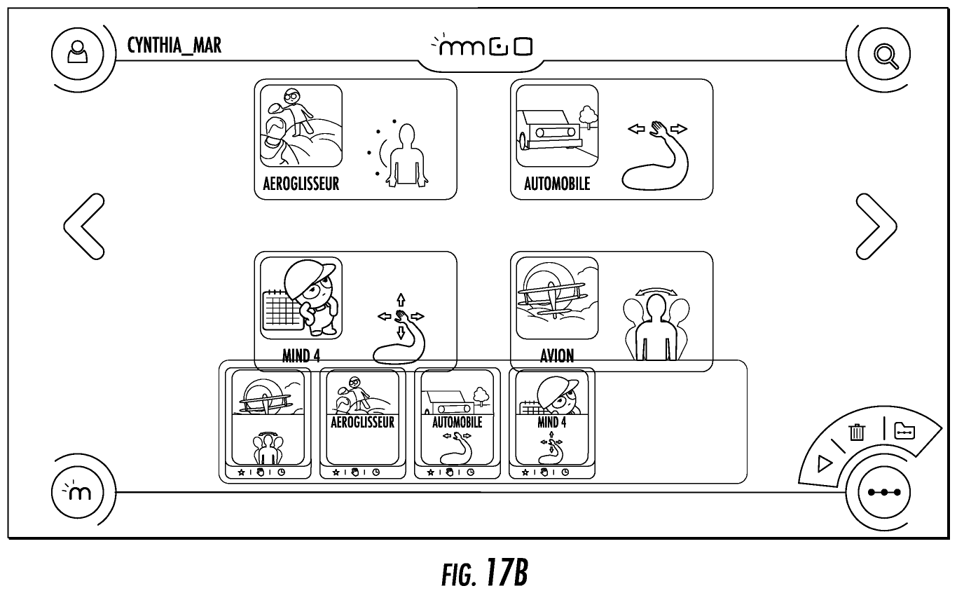

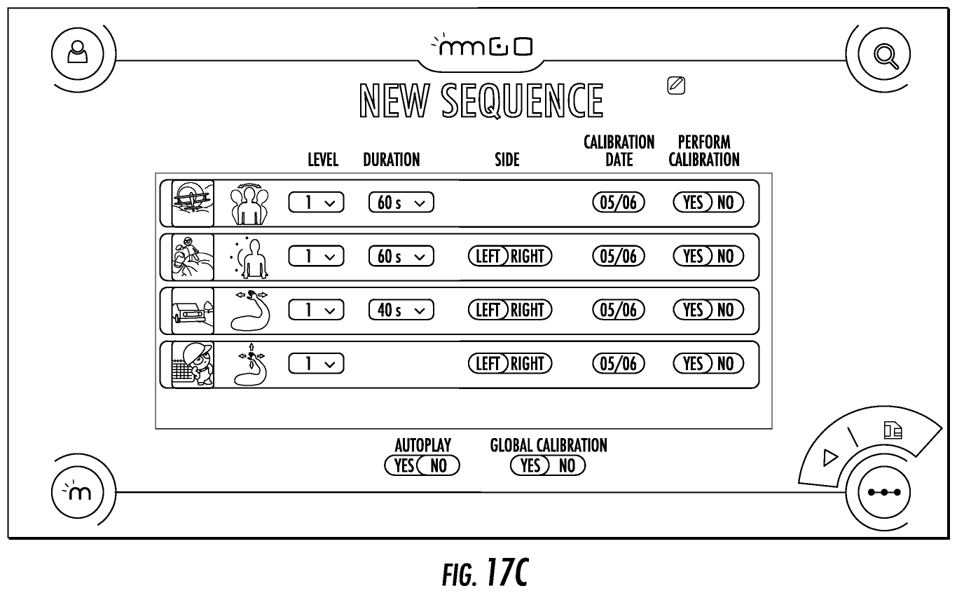

FIGS. 17A-17D show another exemplary, illustrative, non-limiting session flow according to at least some embodiments of the present invention. FIG. 17A shows a screenshot with a plurality of exercises from which a therapist may select. FIG. 17B shows a screenshot with an example exercise sequence for a session. FIG. 17C shows a screenshot with a new sequence of exercises being constructed for a session. FIG. 17D shows a screenshot with a selection of sessions that the user, such as a therapist, can load.

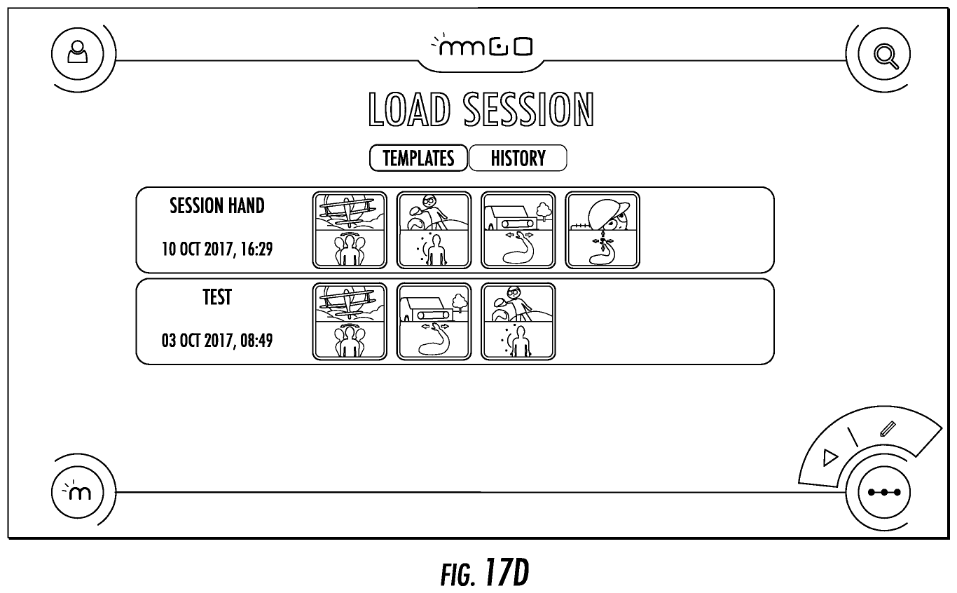

FIGS. 18A and 18B show exemplary, non-limiting screenshots of example games according to at least some embodiments of the present invention. FIG. 18A shows a screenshot of an example game relating to “driving” through an environment. FIG. 18B shows a screenshot of an example game relating to “climbing” through an environment. Of course, many other such games are possible and are contemplated within the scope of the present invention.

While the invention has been described with respect to a limited number of embodiments, it will be appreciated that many variations, modifications and other applications of the invention may be made, including different combinations of various embodiments and sub-embodiments, even if not specifically described herein.

What is claimed is:

1. A system for monitoring a behavior of a user through an interactive computer program, comprising
a. a user movement tracking sensor for generating sensor data of a plurality of movements of the user;
b. a data analysis layer for receiving said sensor data, said data analysis layer further comprising a gesture analysis module and a system calibration module, wherein said system calibration module determines a range of motion of the user for a physical action, and wherein said gesture analysis module decomposes said sensor data into a plurality of gestures, each gesture being calibrated according to said range of motion of the user for the physical action;
c. an interactive computer program layer for receiving said plurality of gestures from said data analysis layer and for determining interaction with said an interactive computer program according to said plurality of gestures; and
d. a computational device for operating said data analysis layer and said interactive computer program layer, and for receiving said sensor data from said user movement tracking sensor.

2. The system of claim 1, wherein said user movement tracking sensor comprises a camera and a depth sensor.

3. The system of claim 1, wherein said user movement tracking sensor comprises a Kinect device.

4. The system of claim 1, wherein said user movement tracking sensor comprises a Leap Motion device.

5. The system of claim 1, wherein said system calibration module comprises a plurality of gesture calibrators, each gesture calibrator analyzing said sensor data to determine said range of motion of the user for the physical action corresponding to a particular gesture.

6. The system of claim 5, wherein said gesture calibrators comprise gesture calibrators for a plurality of gestures selected from the group consisting of trunk gesture, hand gesture, shoulder gesture, leg gesture, finger gesture and arm gesture.

7. The system of claim 5, wherein said system calibration module determines said range of motion of the user for the physical action in a separate calibration process before an interaction begins.

8. The system of claim 7, wherein said gesture analysis module models at least one physical limitation of the user according to said gesture calibrators to form a calibrated gesture and normalizes said calibrated gesture to form a normalized gesture; said gesture analysis module transmitting said normalized gesture to said interaction layer.

9. The system of claim 8, further comprising a device abstraction layer for receiving sensor data from said user movement tracking sensor, for abstracting said sensor data and for transmitting said abstracted sensor data to said data analysis layer.

10. The system of claim 9, further comprising a physical access key, a user data storage for storing user data and a user interface for accessing said user data storage, wherein said computational device operates said user interface; wherein said user interface permits access to said user data storage only if said physical access key is in communication with said computational device.

11. The system of claim 10, wherein said physical access key comprises a dongle, and wherein said physical access key is in communication with said computational device when said physical access key is inserted to a USB (Universal Serial Bus) port of said computational device.

12. The system of claim 1, adapted for therapy of the user through said interactive computer program.

13. The system of claim 1, adapted for diagnosis of the user through said interactive computer program.

14. The system of claim 1, wherein said behavior is selected from the group consisting of a performing physical action, response time for performing said physical action and accuracy in performing said physical action.

15. The system of claim 14, wherein said physical action comprises a physical movement of at least one body part.

16. The system of claim 1, adapted for cognitive therapy of the user through said interactive computer program.

17. The system of claim 16, adapted for performing an exercise for cognitive training.

18. The system of claim 17, wherein said exercise for cognitive training is selected from the group consisting of attention, memory, executive function.

19. The system of claim 18, wherein said system calibration module further determines if the user has a cognitive deficit, such that said system calibration module also calibrates for said cognitive deficit if present.

20. The system of claim 1, wherein said interactive computer program comprises a game.

21. The system of claim 1, wherein said monitoring further comprises decoding said behavior of the user to identify an action taken by the user.

22. The system of claim 1, further comprising a session constructer for enabling a session to be constructed, said session comprising a plurality of game action modules; and a session executer, for executing said session with the user.

23. A method for calibrating the system of claim 1 for a user, the method being performed by a computational device, the method comprising: receiving user movement information for at least one user movement calibration action; calibrating at least one movement of the user from said user movement information; modeling at least one physical limitation of the user according to said user movement information; normalizing at least one gesture according to said modeled physical limitation; and transmitting said normalized gesture to said interactive computer program layer.

24. A method for protecting user information stored in the system of claim 1, the method being performed by a computational device, the method comprising: providing a physical access key and a user data storage for storing user data; detecting whether said physical access key is in communication with said computational device; if said physical access key is in communication with said computational device, permitting access to said user data storage.

25. The method of claim 24, wherein said physical access key is a dongle for insertion to a USB port of the computational device, and wherein said detecting whether said physical access key is in communication with said computational device comprises determining whether said dongle has been inserted to said USB port.

26. The method of claim 25, wherein said permitting access to said user data storage further comprises determining whether said dongle has a valid license stored therein.

27. The method of claim 26, further comprising attempting to validate said computational device through communication with a server, such that access to said user data storage is permitted according to said communication with said server.
